(19) United States
(12) Patent Application Publication
(10) Pub. No.: US 2019/0252063 A1
(43) Pub. Date: Aug. 15, 2019

(54) MONITORING SYSTEM FOR CARE PROVIDER

(71) Applicant: INTERNATIONAL BUSINESS MACHINES CORPORATION, Armonk, NY (US)

(72) Inventors: Michael S. Gordon, Yorktown Heights, NY (US); Jinho Hwang, Ossining, NY (US); Valentina Salapura, Yorktown Hieghts, NY (US); Maja Vukovic, New York, NY (US)

(21) Appl. No.: 15/896,932

(22) Filed: Feb. 14, 2018

(51) Int. Cl. G16H 40/20; G16H 40/63; G06F 15/18; G06Q 50/22

(52) U.S. Cl. CPC G16H 40/20; G06Q 50/22; G06F 15/18; G16H 40/63

(57) ABSTRACT

A method for providing recommendations to a care provider includes receiving, by a monitoring system, environmental information regarding an environment in which a care provider is providing care to a recipient. The environmental information includes interaction data regarding interactions between the care provider and the recipient and entity data regarding entities in the environment. The method includes applying analytic analysis to the environmental information to generate input to a machine learning model. The input includes first features indicative of aspects of the interactions and second features indicative of one or more relations between the entities. The method includes determining a recommendation for the care provider that is predicted to facilitate achieving a goal associated with the recipient by applying the machine learning model to the input. The method includes providing the recommendation by the monitoring system to the care provider.

### BACKGROUND

The present disclosure relates to monitoring a caregiver and providing recommendations. A caregiver may not be sufficiently self-aware or sufficiently trained to provide appropriate care to a care recipient, which may lead to problems in the caregiver's provision of care.

### SUMMARY

According to an embodiment of the present disclosure, a method for providing recommendations to a care provider includes receiving, by a monitoring system, environmental information regarding an environment in which a care provider is providing care to a recipient. The environmental information includes interaction data regarding interactions between the care provider and the recipient and entity data regarding entities in the environment. The method includes applying analytic analysis to the environmental information to generate input to a machine learning model. The input includes first features indicative of aspects of the interactions and second features indicative of one or more relations between the entities. The method includes determining a recommendation for the care provider that is predicted to facilitate achieving a goal associated with the recipient by applying the machine learning model to the input. The method includes providing the recommendation by the monitoring system to the care provider.

According to another embodiment of the present disclosure, a monitoring system includes a recommendation engine. The recommendation engine is configured to receive environmental information regarding an environment in which a care provider is providing care to a recipient. The environmental information includes interaction data regarding interactions between the care provider and the recipient and entity data regarding entities in the environment. The recommendation engine is configured to apply analytic analysis to the environmental information to generate input to a machine learning model. The input includes first features indicative of aspects of the interactions and second features indicative of one or more relations between the entities. The recommendation engine is configured to determine a recommendation for the care provider that is predicted to facilitate achieving a goal associated with the recipient by applying the machine learning model to the input. The monitoring system includes a notification device coupled to the recommendation engine and is configured to provide the recommendation to the care provider.

According to another embodiment of the present disclosure, a computer program product includes a computer readable storage medium having program instructions embodied therewith. The program instructions are executable by a computer to cause the computer to receive environmental information regarding an environment in which a care provider is providing care to a recipient. The environmental information includes interaction data regarding interactions between the care provider and the recipient and entity data regarding entities in the environment. The program instructions are further executable by the computer to cause the computer to apply analytic analysis to the environmental information to generate input to a machine learning model. The input includes first features indicative of aspects of the interactions and second features indicative of one or more relations between the entities. The program instructions are further executable by the computer to cause the computer to determine a recommendation for the care provider that is predicted to facilitate achieving a goal associated with the recipient by applying the machine learning model to the input.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 shows an illustrative block diagram of a system configured to monitor a care provider and to provide a recommendation;

FIG. 2 shows an illustrative block diagram of a recommendation engine that includes a neural network;

FIG. 3 shows a flowchart illustrating aspects of operations that may be performed in accordance with various embodiments; and

FIG. 4 shows an illustrative block diagram of an example data processing system that can be applied to implement embodiments of the present disclosure.

The illustrated figures are only exemplary and are not intended to assert or imply any limitation with regard to the environment, architecture, design, or process in which different embodiments may be implemented. Any optional component or steps are indicated using dash lines in the illustrated figures.

### DETAILED DESCRIPTION

It should be understood at the outset that, although an illustrative implementation of one or more embodiments are provided below, the disclosed systems, computer program product, and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

As used within the written disclosure and in the claims, the terms “including” and “comprising” are used in an open-ended fashion, and thus should be interpreted to mean “including, but not limited to”. Unless otherwise indicated, as used throughout this document, “or” does not require mutual exclusivity, and the singular forms “a”, “an” and “the” are intended to include the plural forms as well, unless the context clearly indicates otherwise.

An engine as referenced herein may comprise of software components such as, but not limited to, data access objects, service components, user interface components, application programming interface (API) components; hardware components such as electrical circuitry, processors, and memory; and/or a combination thereof. The memory may be volatile memory or non-volatile memory that stores data and computer executable instructions. The computer executable instructions may be in any form including, but not limited to, machine code, assembly code, and high-level programming code written in any programming language. The module may be configured to use the data to execute one or more instructions to perform one or more tasks.

Embodiments of the disclosure include a system that determines and provides recommendations to a care provider regarding care of a recipient by the care provider. The system may provide the recommendation via a graphical user interface (GUI) on a smart device, such as a phone or wearable device (e.g., watch) worn or carried by the care provider, on an external speaker, or via earbuds worn by the care provider. In some examples, the system collects and receives sensor data, such as images, video, audio, physiological measurements, and retrieves information (e.g., digital information) that may include blueprints regarding an environment, health information of the recipient, goal information regarding the recipient, and analyzes an environment that includes the care provider and the recipient to determine the recommendations.

In some examples, the recommendations include behavior modification of the care provider. For example, the care provider may be attempting to discipline the recipient, and the system may recognize that the care provider is not being firm enough with the recipient. In this example, the system may recommend that the care provider modify her behavior in order to more effectively discipline the recipient. For example, the system may recommend that the care provider be firmer with the child. As another example, the care provider may be attempting to discipline the recipient, and the system may recognize that the care provider is behaving in a manner that may harm the recipient. In this example, the system may recommend that the care provider modify her behavior so as not to harm the recipient. Alternatively or additionally, the recommendations include actions to avoid injury to the recipient. For example, the recipient may be a child that is too young to safely handle scissors. In this example, the system may detect the presence of scissors in a proximity of the child and may recommend that the care provider move the scissors or the recipient to avoid injury to the recipient.

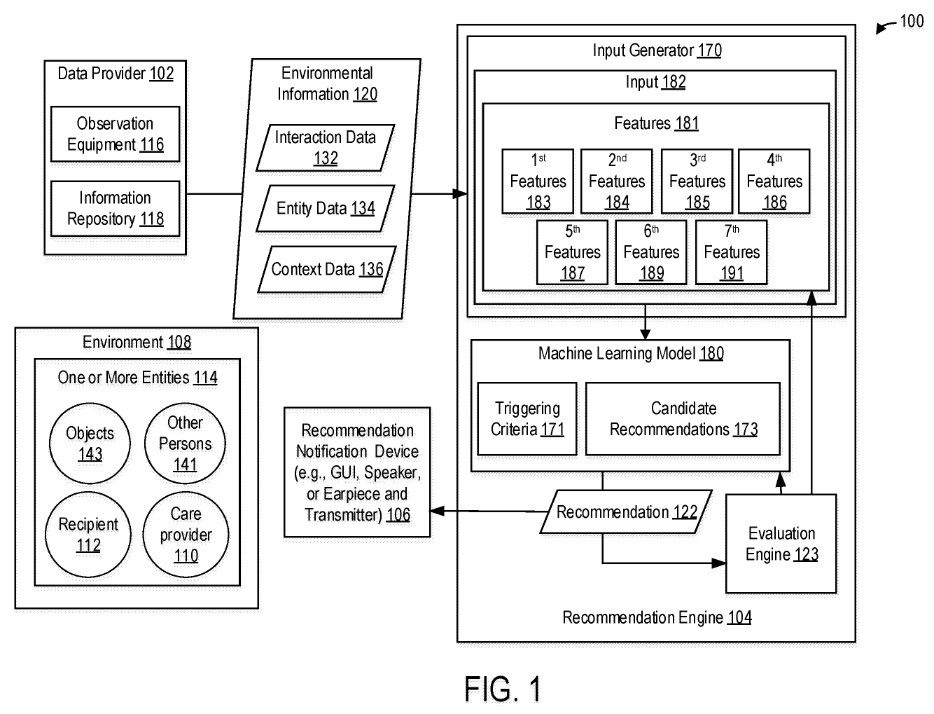

FIG. 1 illustrates an example of a monitoring system 100 configured to provide one or more recommendations to a care provider 110 providing care to a recipient 112, and illustrates an example of an environment 108 in which the care provider 110 is providing care to the recipient 112. The monitoring system 100 includes a recommendation engine 104 and a recommendation notification device 106. The monitoring system 100 illustrated in FIG. 1 also includes a data provider 102. Although the monitoring system 100 illustrated in FIG. 1 includes the data provider 102, in other examples, the monitoring system 100 does not include the data provider 102, or includes the observation equipment 116 (e.g., sensors) and not the information repository 118. In addition to the care provider 110 and the recipient 112, the environment 108 may include one or more entities 114, such as objects 143 or persons 141 other than the care provider 110 and the recipient 112.

One or more components of the monitoring system 100 may be located in or near the environment 108. For example, the observation equipment 116 may be located in or near the environment 108 to enable the observation equipment 116 to provide environmental information 120 regarding the environment 108 as described in more detail below. Alternatively or additionally, one or more components of the monitoring system 100 may be located remotely from the environment 108. For example, the recommendation engine 104 may be deployed remotely from the observation equipment 116 (e.g., such as in a server or processor located in a hub in a school).

In some examples, the care provider 110 is a teacher, the recipient 112 is a student, and the environment 108 in which the care provider 110 is providing care to the recipient 112 is a classroom. In other examples, the care provider 110 is a parent and the recipient 112 is a child of the parent. In other examples, the care provider 110 is a babysitter or nanny and the recipient 112 is a child under the care and supervision of the babysitter or nanny. In other examples, the care provider 110 is a caregiver for seniors or elderly people and the recipient 112 is a senior or elderly person under the care and supervision of the caregiver.

The data provider 102 is configured to provide environmental information 120 regarding the environment 108. In one example, the environment 108 is the area surrounding the recipient 112 and the care provider 110. In the example illustrated in FIG. 1, the data provider 102 includes the observation equipment 116. The observation equipment 116 includes one or more sensors and is configured to monitor the environment 108, including entities within the environment 108. The observation equipment 116 may include audio capturing, video capturing, or audio visual capturing equipment. To illustrate, the observation equipment 116 may include one or more cameras, one or more microphones, or both, that record or capture interactions between the care provider 110 and the recipient 112. Additionally or alternatively, the observation equipment 116 may include physiological sensing or measurement equipment that provides physiological data regarding physiological aspects of the care provider 110, the recipient 112, or both. To illustrate, the observation equipment 116 may include a wearable device, such as a watch or bracelet, that includes a temperature sensor, a perspiration sensor, blood pressure sensor and/or a heart rate sensor that is worn by the care provider 110 or the recipient 112 and that provides temperature, perspiration, blood pressure and/or heart rate information regarding the care provider 110 or the recipient 112 that is wearing the observation equipment 116.

The environmental information 120 includes interaction data 132 regarding current and previous interactions between the care provider 110 and the recipient 112, and includes entity data 134 regarding one or more entities in the environment 108. The environmental information 120 may additionally include context data 136.

The interaction data 132 may be in the form of audio, visual, or audio-visual data that represents interactions between the care provider 110 and the recipient 112. The interaction data 132 may be provided by the observation equipment 116. For example, the interaction data 132 may correspond to or include audio, video, or audio-visual data of interactions between the care provider 110 and the recipient 112 that are captured by one or more cameras or microphones of the observation equipment 116.

The entity data 134 is data regarding one or more entities in the environment 108. The one or more entities in the environment 108 may include persons or objects. For example, the one or more entities may include the care provider 110, the recipient 112, and other persons, such as other children, or other care providers in the environment 108.

The entity data 134 may regard physiological aspects of the care provider 110 or the recipient 112. To illustrate, in an example in which the one or more entities correspond to (or include) the care provider 110 and the recipient 112, the entity data 134 may include data regarding real time physiological aspects or attributes of the care provider 110 or the recipient 112. The physiological aspects or attributes may include temperature, perspiration, blood pressure and/or heart rate. For example, the observation equipment 116 may include a wearable device, such as a watch or bracelet, that includes a temperature sensor, a perspiration sensor, a blood pressure sensor and/or a heart rate sensor that is worn by the care provider 110 or the recipient 112 and that provides temperature, perspiration, blood pressure and/or heart rate information regarding the care provider 110 or the recipient 112 that is wearing the observation equipment.

Additionally or alternatively, the entity data 134 may regard background or context regarding the care provider 110. To illustrate, in an example in which the one or more entities correspond to or include the care provider 110, the entity data 134 may additionally or alternatively include personality data, historical data of engagement with recipients, health data, illness data, or any combination thereof, regarding the care provider 110. In this example, the entity data 134 may be received from an information repository, such as the information repository 118.

Additionally or alternatively, the entity data 134 may regard background or context regarding the recipient 112. To illustrate, in an example in which the one or more entities correspond to or include the recipient 112, the entity data 134 may additionally or alternatively include personality data, preferred language for communicating with the recipient 112, current goals (e.g., learning to read, potty training), historical data of responses to types of discipline, health data, illness data, special needs (e.g., due to attention deficit hyperactive disorder or autism), sibling information, age information, or any combination thereof, regarding the recipient 112. In this example, the entity data 134 may be received from an information repository, such as the information repository 118. The information repository 118 may correspond to a computer or server that stores all or some of the entity data 134.

Additionally or alternatively, the entity data 134 may regard aspects of objects in the environment 108. To illustrate, in examples in which the one or more entities include objects in the environment 108, the entity data 134 may include data indicating a location of the object or a type of the object. For example, the object may include a hot water heater, and the entity data 134 may include a blueprint from which the existence and location of the hot water heater may be discerned or learned. In this example, the entity data 134 is retrieved from the information repository 118 that stores the blueprint. As another example, the object may include scissors, and the entity data 134 may include image or video data (of the environment 108) that includes one or more images of the scissors. In this example, the entity data 134 includes data provided by the observation equipment 116.

Additionally or alternatively, the entity data 134 may regard aspects of other persons in the environment 108. For example, the entity data 134 may include data that indicates an age of other persons in the environment 108 such as other children at a day care center or school.

The environmental information 120 may include context data 136. The context data 136 may indicate a context regarding the environment 108. For example, the context data 136 may include a location of the environment 108, a current time, or a setting of the environment 108 (e.g., playroom or classroom). The context data 136 may be provided by the information repository 118.

The recommendation engine 104 includes an input generator 170 configured to apply analytic analysis to the environmental information 120 to generate input 182 for a machine learning model 180. The input 182 may correspond to a feature vector of features 181. Each of the features 181 is an individual measurable property or characteristic that the machine learning model 180 uses to determine the recommendation 122, and the input generator 170 may be configured to generate the input 182 by performing pattern representation and feature measurement based on the environmental information 120.

The analytic analysis may include object detection, object tagging, parsing and matching, and determining entities and relations. The features 181 include first features 183 indicative of aspects of the interactions between the care provider 110 and the recipient 112, and may be determined by applying analytic analysis to the interaction data 132 and/or to the entity data 134.

The aspects of the interactions between the care provider 110 and the recipient 112 may include an interaction type. For example, interaction types may include a disciplinary interaction type, a social interaction type, an instructive interaction type, or an interrogatory interaction type. In this example, the first features 183 may include content or substance of communication between the care provider 110 and the recipient 112. To illustrate, keyword phrases such as “I told you not to,” “you are not allowed,” or “this is the second time I told you,” may correspond to features indicative of a disciplinary interaction type. In this example, the input generator 170 is configured to apply analytic analysis to the interaction data 132 to determine the presence of keyword phrases, and may populate the feature vector based on detection of the keyword phrases.

As another example, aspects of the interactions between the care provider 110 and the recipient 112 may include state of mind of the care provider 110 or the recipient 112 during the interactions. In this example, the first features 183 may include features that map to emotion or state of mind. To illustrate, the first features 183 may include features of speech indicative of the state of mind, such as tone, volume or anger. In this example, the input generator 170 is configured to process audio data of the interaction data 132 from the observation equipment 116 to determine the features, such as tone, volume or anger. Alternatively or additionally, in some examples, the first features 183 may include features of posture, such as stiff, having crossed arms, or standing over the recipient 112. In this example, the input generator 170 is configured to process video data from the interaction data 132 to determine measurements of the posture of either the care provider 110 or the recipient 112. Alternatively or additionally, in some examples, the first features 183 may include physiological features, such as temperature, perspiration, or blood pressure of the care provider 110 or the recipient 112. In this example, the input generator 170 is configured to process physiological data of the entity data 134 from the observation equipment 116 to determine measurements of the temperature, perspiration or blood pressure. For instance, the care provider 110 might be getting upset as indicated by an increase in her blood pressure.

The features 181 include second features 184 indicative of one or more relations between the entities. The relations may include relations between objects (e.g., first entities) and the recipient 112 (e.g., a second entity). To illustrate, the second features 184 may include a distance between the recipient 112 and objects in the environment 108. The objects may be identified in the environment 108 based on the entity data 134. For example, the entity data 134 may include video data from the observation equipment 116 as described above, and the video data may capture an image of scissors in the environment 108. In this example, the input generator 170 may process the video data to determine a feature corresponding to a distance (e.g., a relation) between the scissors and the recipient 112.

As another example, the relations may include relations between the recipient 112 and one or more other persons in the environment 108. The entities—e.g., the recipient 112 and the one or more other persons in the environment 108—may be identified based on the entity data 134. For example, the entity data 134 may include video data from the observation equipment 116 as described above, and the video data may capture an image of the recipient 112 and the other child. In this example, the input generator 170 may process the video data to identify the recipient 112 and the other child in the video data, and determine a relation that the recipient is in contact with the other child. In this example, the relations include a relation of physical contact between the recipient 112 and another child in the environment 108; however, in other examples, the relations between the recipient and other persons in the environment 108 may include other types of relations, such as “yelling at,” “throwing an object at,” or “hitting at.”

The features 181 may include third features 185 indicative of behavior or state of mind of the recipient 112 that does not fall within the first features 183 and the second features 184. For example, the recipient 112 may be yelling, but may not be yelling at another person or entity. Thus, the recipient yelling in this example may not correspond to an aspect of interactions between the care provider 110 and the recipient 112 or a relation between the recipient 112 and another entity, and thus may not fall within the first features 183 and the second features 184. The third features 185 are determined based on the entity data 134. To illustrate, the recipient 112 may be crying, and the observation equipment 116 may capture audio data of the recipient 112 crying. In this example, the input generator 170 is configured to process audio data of the entity data 134 from the observation equipment 116 to determine feature measurements corresponding to particular frequencies or patterns of sound that are produced by the recipient 112 and that are indicative of crying. Additionally or alternatively, the particular frequency or patterns that are indicative of crying may also be indicative of a sad, frustrated, hungry, tired, or angry state of mind of the recipient 112. Thus, the feature measurements corresponding to the particular frequencies or patterns of sound that are produced by the recipient 112 may also be indicative of a state of mind of the recipient 112.

As another example, the third features 185 may include features indicative of physiological aspects of the recipient 112. To illustrate, the observation equipment 116 may provide the entity data 134 indicative of temperature, perspiration, blood pressure, and/or heart rate, and the temperature, perspiration, blood pressure and/or heart rate information may be indicative of a state of mind of the recipient 112. For example, a particular pattern of temperature, perspiration, blood pressure, and/or heart rate may be correlated with the recipient being angry, hungry, or tired. In these examples, the third features 185 may include measurements of the various physiological aspects that may be indicative of a state of mind of the recipient 112.

The features 181 may include fourth features 186 indicative of a state of mind of the care provider 110 that does not fall within the first features 183 and the second features 184. For example, a state of mind of the care provider 110 may include a tired state of mind, and the fourth features 186 may include an amount of time that the care provider 110 has her eyes closed, a movement tempo, a speech tempo, data regarding how many hours the care provider 110 slept during a predetermined period (e.g., the night before), data regarding how well the care provider 110 slept during a predetermined period (e.g., the night before), or a combination thereof. For example, the care provider 110 may be sitting at her desk with her eyes closed, and the fourth features 186 may include a length of time that the care provider 110 has her eyes closed. In this example, the input generator 170 is configured to process video data of the entity data 134 from the observation equipment 116 to determine feature measurements corresponding to an amount of time that the care provider 110 has her eyes closed.

The features 181 may include fifth features 187 indicative of background or context regarding the recipient 112. To illustrate, the entity data 134 may include personality data, historical data of responses to types of discipline, goals, health data, illness data, special needs, or any combination thereof regarding the recipient 112, and the fifth features 187 may include features indicative of the personality, goals, responses to types of discipline, health data, illness data, special needs, or any combination thereof.

The features 181 may include sixth features 189 indicative of the entities. For example, the sixth features 189 may include aspects (e.g., the existence or location) of an object. To illustrate, the sixth features 189 may include the existence or location of a hot water heater in the environment 108. In this example, the entity data 134 may include a blueprint from which the existence and location of the hot water heater may be discerned or learned as described above. In this example, the entity data 134 is retrieved from the information repository 118 that stores the blueprint, and the input generator 170 processes the blueprint to determine features of the sixth features 189 that indicate a location of the hot water heater. As another example, the object may include scissors, and the entity data 134 may include image or video data (of the environment 108) that captures one or more images of the scissors. In this example, the entity data 134 includes data provided by the observation equipment 116, and the input generator 170 processes the video data to determine a location of the scissors. As another example, the sixth features 189 may regard aspects of other persons in the environment 108. To illustrate, the sixth features 189 may include the age and number of other children in the environment 108. In this example, the entity data 134 may include age information regarding the other children in the environment 108, and the input generator 170 may process the entity data 134 to determine features of the sixth features 189 that indicate an age and number of the other children in the environment 108.

The features 181 may include seventh features 191 indicative of a context regarding the environment 108. For example, the seventh features may be indicative of a location of the environment 108, a current time, or a setting of the environment 108 (e.g., playroom or classroom).

The recommendation engine 104 is configured to apply a machine learning model 180 to the input 182 to determine the recommendation 122 for the care provider 110 that is predicted to facilitate achieving a goal associated with the recipient 112. The goal may correspond to ameliorating behavior of the recipient 112 or learning a new skill by the recipient 112. Additionally or alternatively, the goal may be directed to safety of the recipient 112. The recommendation 122 may be selected from a plurality of candidate recommendations 173.

The machine learning model 180 may be implemented as a bayesian model, a clustering model (e.g., k-means), an artificial neural network (e.g., perceptron, back-propagation, hopfield, radial basis function network), a deep learning network (e.g., deep boltzmann machine, deep belief network, convolutional neural network), and may include supervised learning, unsupervised learning, semi-supervised learning, and reinforcement learning.

The machine learning model 180 is configured to determine, select, or provide the recommendation 122 responsive to triggering criteria 171. In some examples, the triggering criteria 171 include detection of a context or pattern corresponding to a particular state of the care provider 110. For example, the machine learning model 180 may be configured to provide a recommendation 122 to the care provider 110 when the machine learning model 180 recognizes a context or pattern corresponding to the care provider 110 being overly tired or angry. To illustrate, the machine learning model 180 may be configured to determine that the care provider 110 is tired based on the fourth features 186, and the determination that the care provider 110 is tired may trigger the machine learning model 180 to provide the recommendation 122. In this example, the recommendation 122 may be to take a break and to let the care provider know that they are tired, so that the care provider 110 can rest and return in a more alert state, thereby enabling the care provider 110 to provide improved care.

As another example, the machine learning model 180 may be configured to provide the recommendation 122 to the care provider 110 when the machine learning model 180 recognizes a pattern corresponding to the care provider 110 exhibiting a particular psychological characteristic during interaction with the recipient 112. To illustrate, the machine learning model 180 may be configured to determine, based on the first features 183, a level of calmness of the care provider 110 while the care provider 110 is disciplining the recipient 112. In this example, the machine learning model 180 may be configured to provide the recommendation 122 when the level of calmness satisfies a threshold. For example, the machine learning model 180 may determine, based on the first features 183 (e.g., physiological attributes of the care provider 110) that the care provider 110 is not sufficiently calm while disciplining the recipient 112, and may recommend that the care provider 110 soften their approach to calm down in order to prevent the situation from getting out of control. Thus, the monitoring system 100 may prevent disciplinary situations from getting out of control by monitoring the behavior or state of mind of the care provider 110 and recommending behavior modification (e.g., to calm down) before disciplining the recipient 112 in an inappropriate fashion.

As another example, the triggering criteria 171 may correspond to detection of a context or pattern corresponding to particular behavior of the recipient 112. For example, the particular behavior may correspond to misbehavior of the recipient 112, and the machine learning model 180 may be configured to provide the recommendation 122 to the care provider 110 when the machine learning model 180 recognizes a context or pattern corresponding to misbehavior of the recipient 112 above a predetermined threshold. In these examples, the candidate recommendations 173 may include different types of disciplinary action (e.g., time-out, send to principal), different types of disciplinary approaches (e.g., positive discipline, gentle discipline, boundary-based discipline, behavior modification, emotion coaching), or both.

To illustrate, the recipient 112 may be biting another child. In this example, the second features 184 may include a relation that the recipient 112 is biting another child. The machine learning model 180 may determine that the recipient 112 biting another child constitutes misbehavior, and may trigger the recommendation 122. In these examples, the recommendation 122 may include suggesting an activity to redirect the recipient 112 or identifying a corrective discipline to be applied by the care provider 110 such as separating the children and disciplining the biter.

In some examples, the machine learning model 180 is configured to consider a health or history of the recipient 112 or the care provider 110 in determining the recommendation 122. To illustrate, the recipient 112 may suffer from asthma that is triggered by stress. In this example, the fifth features 187 may indicate that the recipient 112 suffers from stress-induced asthma and the machine learning model 180 may be configured to determine a disciplinary recommendation that is designed to reduce (or not increase) stress. To illustrate, based at least in part on the fifth features 187 indicating that the recipient 112 suffers from stress-induced asthma, the machine learning model 180 may determine to recommend a gentle disciplinary approach as opposed to a harsher disciplinary approach and monitor its effectiveness.

Additionally or alternatively, in some examples, the machine learning model 180 is configured to determine the recommendation 122 based at least in part on a prohibited discipline (e.g., from parents). For example, the fifth features 187 may indicate that the parents of the recipient 112 prohibit use of a certain type of disciplinary approach. To illustrate, the fifth features 187 may indicate that the parents of the recipient 112 prohibit use of physical discipline, or time-out. In this example, the machine learning model 180 is configured to determine a disciplinary recommendation that does not employ physical discipline and does not use time-out.

Additionally or alternatively, in some examples, the machine learning model 180 is configured to determine the recommendation 122 based at least in part on a preferred disciplinary style to be employed as indicated by parents of the recipient 112 or preferred approaches from other parents of similar cohorts of day care recipients. For example, the parents of the recipient 112 may be employing a certain type of instructional or disciplinary approach at home. In order to maintain consistency, the parents may desire that the recipient 112 be disciplined using the same type of disciplinary approach used by the parents. In this example, the entity data 134 may include background or context that indicates the particular type of disciplinary approach the parents want to be used, the fifth features 187 may indicate the particular approach that the parents want to be used, and the machine learning model 180 may be configured to determine a disciplinary recommendation based at least in part on the particular type of disciplinary approach indicated by the fifth features 187 such that the recommendation 122 recommends a type or form of discipline that is consistent with the type of discipline the parents use with the recipient 112.

As another example, the triggering criteria 171 may correspond to detection of a context or pattern corresponding to effectiveness of disciplinary action. For example, the machine learning model 180 may be configured to provide the recommendation 122 to the care provider 110 when the machine learning model 180 determines that disciplinary action is not sufficiently effective. In these examples, the recommendation 122 may be to modify a behavior of the care provider 110 to make the disciplinary action more effective. In these examples, the candidate recommendations 173 may include different types of behavior modification (e.g., be firmer, calm down, or stop yelling). As an example, the machine learning model 180 may be configured to determine that the care provider 110 is disciplining the recipient 112 based on the input 182, determine a behavior or state of mind of the care provider 110 and/or the recipient 112 based on the input 182, and provide a recommendation to the care provider 110 to facilitate more effective discipline. To illustrate, the machine learning model 180 may determine that the care provider 110 is disciplining the recipient 112 for climbing on a hot water heater while the recipient 112 is still on the hot water heater. In this example, the machine learning model 180 may determine that the recipient 112 is not receptive to the discipline based on the continued behavior of the recipient 112 in climbing the hot water heater (or not coming down from the hot water heater). In this example, the machine learning model 180 may also determine that the care provider 110 is not being firm enough with the recipient, and may recommend that the care provider 110 be firmer.

In some examples, the machine learning model 180 is configured to consider the behavior of the recipient 112 in context when determining whether to recommend discipline and what type of disciplinary action to take. The context may include a setting or location of the environment 108. For example, behavior of the recipient 112 that is acceptable on a playground may be unacceptable (and thus warrant discipline) when exhibited in a classroom. To illustrate, as described above, the features 181 may include seventh features 191 that indicate a setting of the environment 108, and the machine learning model 180 may be configured to determine whether discipline is recommended and/or what type of discipline to recommend based in part on the setting. As another example, the context may include aspects of other persons in the environment 108. For example, a particular interaction between the recipient 112 and another child may be acceptable when the interaction is between the recipient 112 and a sibling of the recipient 112, and may be unacceptable when the interaction is between the recipient 112 and a non-family member. In this example, the features 181 may include sixth features 189 that indicate whether another person with whom the recipient 112 is interacting is a sibling of the recipient 112, and the machine learning model 180 may be configured to determine whether discipline is recommended responsive to the interaction, and/or what type of discipline to recommend responsive to the interaction, based in part on whether the interaction is with a sibling of the recipient 112.

The recommendation engine 104 may be configured to learn about behavior patterns of the recipient 112 and what actions/responses of the care provider 110 are most effective at achieving a goal as described above or most effective at disciplining the recipient 112. The recommendation engine 104 may modify the features 181 or the machine learning model 180 so that the features 181 include features that map to certain actions/responses of the care provider 110 that are most effective for disciplining the recipient 112, and so that the machine learning model 180 accounts for the patterns of the recipient 112 and the effectiveness of the actions/responses of the care provider 110. In these examples, the recommendation engine 104 may employ reinforcement learning training. For example, the recommendation engine 104 may include an evaluation engine 123 to evaluate the effect of discipline to certain behavior of the recipient 112. The evaluation engine 123 may provide feedback that reflects the effectiveness of the discipline to the machine learning model 180. The evaluation engine 123 determines the feedback by evaluating or analyzing the action of the care provider 110 and the effect on the recipient 112. The machine learning model 180 may be trained (e.g., modified) based on the feedback. Additionally or alternatively, the evaluation engine 123 may determine whether to provide a reward (e.g., positive reinforcement) to the care provider 110 based on how effective the care provider 110 is at disciplining the recipient 112 or following the recommendation 122. Thus, the recommendation engine 104 may be configured to learn about patterns of the recipient 112 and what actions/responses are most effective, and may account for the patterns and effectiveness when determining the recommendation 122.

In some examples, the recommendation engine 104 may track the rewards to determine whether to replace the care provider 110. For example, the recommendation engine 104 may maintain a cumulative tally of the rewards, and may recommend to a responsible entity (e.g., parents or school administrator) to replace, reassign, or remove the care provider 110.

Thus, the machine learning model 180 may be configured to process the features 181 to determine whether the recipient 112 should be disciplined, and, when discipline is recommended, what particular type of discipline to apply based on the environmental information 120 and based on learned patterns and effectiveness of the discipline.

As another example, the triggering criteria 171 may correspond to detection of a context or pattern corresponding to good behavior or accomplishing a goal. For example, the machine learning model 180 may be configured to provide the recommendation 122 to the care provider 110 when the machine learning model 180 determines that the recipient 112 has engaged in good behavior. In these examples, the recommendation 122 may be to reward the child by proving a reward or giving positive reinforcement. To illustrate, the fifth features 187 may indicate that the recipient 112 is in a stage in which she is learning to read, and the machine learning model 180 may determine, based on the fifth features 187, that the recipient 112 successfully read a sentence or a chapter in a book. In this example, the machine learning model 180 may determine to provide a recommendation 122 to the day care provider 110 to reward the recipient 112.

As another example, the triggering criteria 171 may correspond to detection of a context or pattern corresponding to an object presenting a sufficiently high risk of danger to the recipient 112. In this example, the recommendation 122 is directed to a safety recommendation. To illustrate, the machine learning model 180 may be configured to determine, for one or more objects detected in the environment 108 and based on the second features, the sixth features, or both, a risk of injury of the object to the recipient 112. For example, the sixth features may indicate the existence of a pair of scissors in the environment 108, and the second features 184 may indicate that the recipient 112 is at a particular distance from the pair of scissors. In this example, the machine learning model 180 may determine that the risk of injury that the scissors present to the recipient 112 at the particular distance exceeds a threshold. The threshold may depend on the age of the recipient 112 or, in this example, the type of scissors (as safety scissors do not pose the same danger threat as kitchen scissors). Based on the machine learning model 180 determining that the risk of injury that the object (e.g., the pair of scissors) presents to the recipient 112 satisfies a threshold, the machine learning model 180 is configured to recommend that the care provider 110 move the object or the recipient 112. As another example, the machine learning model 180 may determine, based on the second features 184 and the third features 185, that the recipient 112 is being offered peanuts and that the recipient 112 is allergic to peanuts. The machine learning model 180 may determine that the recipient 112 is being offered peanuts and alert the care provider 110 to not offer peanuts to the child. As another example, the machine learning model 180 may determine, based on the second features 184 that the recipient 112 is experiencing a medical situation, (e.g. allergic reaction) and may process the input 182 to determine a recommendation 122 that includes an alert to sensitivities of the recipient 112 or provide an alert to medical authorities if necessary.

The machine learning model 180 may employ or include a bayesian model to determine recommendations 122 directed to safety. To illustrate, the input 182 may correspond to a feature vector X=(x₁, x₂, . . . , x_{d})^{T}, where d is a number of the features 181 and T represents transposition. For example, the feature x₁ may indicate the existence of scissors in the environment 108 and the feature x₂ may indicate a distance between the scissors and the recipient 112. The bayesian model may be configured to assign the feature vector X to one of c categories in Ω={ω₁, ω₂, . . . , ω_{c}}. To illustrate, the c categories may include a first category of ‘do nothing’ and a second category of ‘move the scissors’. To assign the feature vector X to one of the c categories, the bayesian model is configured to determine prior probabilities according to the following Equations 1-3, where P(ωᵢ) are prior probabilities, P(X|ωᵢ) are class-conditional probabilities, and α(X) corresponds to an optimal decision rule for minimizing the risk:

$$\begin{matrix}P ( X ) = {\sum }_{i = 1}^{c} P ( X \vert {ω}_{i} ) P ( {ω}_{i} ) & Equation 1 \\ P ( {ω}_{i} \vert X ) = \frac{P ( X \vert {ω}_{i} ) P ( {ω}_{i} )}{P ( X )} & Equation 2 \\ α ( X ) = {arg min}_{{ω}_{i}} R ( {ω}_{i} \vert X ) & Equation 3\end{matrix}$$

Thus, the monitoring system 100 is configured to detect objects or situations that present a sufficiently high risk of danger to the recipient 112, and to provide a recommendation 122 to a care provider 110 to address the risk.

The recommendation notification device 106 may correspond to a device to be worn by the care provider 110 (e.g., a watch or earpiece), a device carried by the care provider 110 (e.g., a smart phone), or an alarm system. When the monitoring system 100 determines the recommendation 122, the monitoring system 100 (e.g., the recommendation engine 104) communicates (e.g., via a transmitter) the recommendation 122 to the recommendation notification device 106. For example, the monitoring system 100 may be located within a near field communication (NFC) range of the recommendation notification device 106, and the monitoring system 100 may transmit data representing the recommendation 122 to the recommendation notification device 106 via NFC capability.

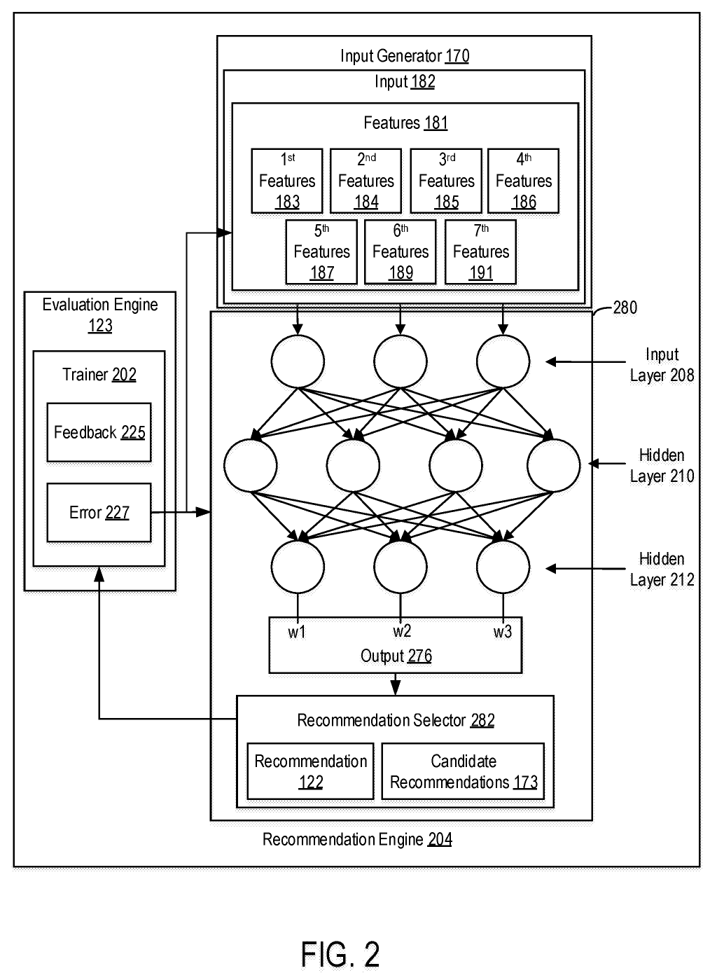

FIG. 2 illustrates an example recommendation engine 204 that includes a neural network 280 implementation of the machine learning model 180 of FIG. 1. The recommendation engine 204 is an example implementation of the recommendation engine 104 of FIG. 1. However, the recommendation engine 104 of FIG. 1 may be implemented using different or alternative aspects. For example, the recommendation engine 104 may be implemented using a machine learning model additional or alternative to a neural network. The neural network 280 of FIG. 2 may correspond to a multilayer perceptron. The neural network 280 of FIG. 2 includes an input layer 208 (e.g., a visible layer) configured to receive the features 181. The neural network 280 of FIG. 2 also includes a hidden layer 210 and a hidden layer 212. Although the neural network 280 of FIG. 2 is illustrated as including two hidden layers, in other examples, the neural network 280 includes more than or less than two hidden layers.

Each node in the hidden layers 210 and 212 is a neuron that maps inputs to the outputs by performing linear combination of the inputs with the node's network weight(s) and bias and applying a nonlinear activation function. One or more nodes in a hidden layer (e.g., the hidden layer 210) may be used to determine triggering criteria (e.g., the triggering criteria 171 described above with reference to FIG. 1). For example, one or more nodes in the neural network 280 may be used to detect a pattern corresponding to one or more of the triggering criteria 171 described above with reference to FIG. 1, and the output 276 may be provided to the recommendation selector 282 when responsive to the neural network determining the triggering criteria. The hidden layer 212 may correspond to an output layer, and a number of nodes in the output layer may correspond to a number of classes or categories of candidate recommendations, such as the candidate recommendations 173 of FIG. 1. For example, the recommendation 122 may be selected from a set of N categories of the candidate recommendations 173, and the number of nodes in the output layer may therefore also include N different recommendations. The output 276 includes a plurality of weights w1, w2, and w3. Although the output 276 is illustrated as including three output weights, in other examples, the output 276 includes more than or less than three output weights (e.g., the output 276 may include a number of output weights corresponding to a number of the set of N candidate recommendations). The weights w1, w2, and w3 may be associated with different recommendations of the candidate recommendations 173 and may be provided to a recommendation selector 282. For example, the categories of candidate recommendations 173 may include N=3 categories. In this example, the first weight w1 may be associated with a first recommendation, the second weight w2 may be associated with a second recommendation, and the third weight w3 may be associated with a third recommendation. The recommendation selector 282 may determine which of the candidate recommendations 173 to use as the recommendation 122 based on which of the weights w1, w2, or w3 is greatest.

The recommendation engine 204 of FIG. 2 includes a trainer 202 configured to train the neural network 280 of FIG. 2 using feedback 225. The feedback 225 reflects the results of an action of the care provider 110 or the recommendation 122. The feedback 225 is based on information provided to or by the evaluation engine 123. The trainer 202 may be configured to perform a back-propagation algorithm based on the feedback 225. The back-propagation may include a backward pass through the neural network 280 that follows a forward pass through the neural network 280. For example, in the forward pass, the outputs 276 corresponding to given inputs (e.g., the features 181) are evaluated. In the backward pass, partial derivatives of the cost function with respect to the different parameters are (e.g., the error 227 is) propagated back through the neural network 280. The network weights can then be adapted using any gradient-based optimization algorithm. The whole process may be iterated until the network weights have converged.

Although FIG. 2 illustrates an example of the neural network 280 as a multiplayer perceptron, in other examples, the neural network 280 is implemented as a Restricted Boltzmann machine or a Deep Belief Network. Additionally, although FIG. 2 illustrates an example of the machine learning model 180 of FIG. 1 as a neural network, in other examples, the machine learning model 180 of FIG. 1 may be implemented using a model other than a neural network.

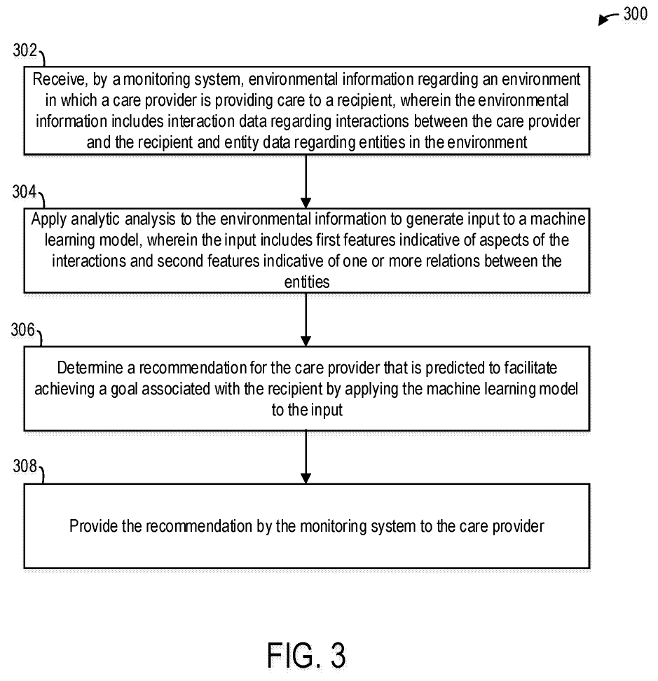

With reference to FIG. 3, a method 300 of providing a recommendation is illustrated. One or more aspects of the method 300 may be performed by one or more components of the monitoring system 100 of FIG. 1 (e.g., the recommendation engine 104) or the recommendation engine 204 of FIG. 2. Thus, one or more aspects of the method 300 may be computer-implemented.

The method 300 includes receiving, at 302, by a monitoring system, environmental information regarding an environment in which a care provider is providing care to a recipient. For example, the recommendation engine 104 of FIG. 1 or the recommendation engine 204 of FIG. 2 may receive the environmental information 120 described above with reference to FIGS. 1 and 2. The environmental information includes interaction data regarding interactions between the care provider and the recipient and entity data regarding entities in the environment. The entities in the environment may include the care provider, the recipient, other persons in the environment, or objects in the environment. In some examples, the interaction data corresponds to the interaction data 132 described above with reference to FIGS. 1 and 2, and the entity data corresponds to the entity data 134 of FIGS. 1 and 2. The monitoring system may receive the environmental information from a data provider, such as the data provider 102 of FIG. 1. In some examples, the monitoring system may receive the environmental information from observation equipment, such as the observation equipment 116 of FIG. 1, that captures the environmental information.

As an example, the observation equipment 116 may include audio recording, video recording, or audio video recording equipment, and may provide audio data, video data, or audio visual data of the environment to the monitoring system. Thus, in some examples, the environmental information corresponds to audio, visual, or audio visual data, and the monitoring system receives audio, visual, or audio visual data from the audio, video, or audio visual equipment. As another example, the observation equipment 116 may include physiological measurement equipment as described above with reference to FIG. 1. In some examples, the environmental information includes context data, such as the context data 136 described above with reference to FIG. 1.

The method 300 additionally includes, at 304, applying analytic analysis to the environmental information to generate input to a machine learning model. For example, the analytic analysis may include object detection, object tagging, parsing and matching, and determining entities and relations as described above with reference to FIG. 1. The input may correspond to the input 182 described above with reference to FIG. 1, and the machine learning model may correspond to the machine learning model 180 of FIG. 1 or the neural network 280 of FIG. 2. The input 182 includes first features indicative of aspects of the interactions and second features indicative of one or more relations between the entities. In some examples, the first features correspond to the first features 183 described above with reference to FIGS. 1 and 2, and the second features correspond to the second features 184 described above with reference to FIGS. 1 and 2.

The aspects of the interactions may include aspects of interactions described above with reference to FIG. 1. The one or more relations may include relations between the care provider and the recipient, between the recipient and other persons in the environment, or relations between the recipient and objects in the environment as described above with reference to FIG. 1.

The method 300 additionally includes determining, at 306, a recommendation for the care provider that is predicted to facilitate achieving a goal associated with the recipient by applying a machine learning model to the input. The recommendation may correspond to the recommendation 122 described above with reference to FIGS. 1 and 2. The goal may correspond to any one or more of the goals described above with reference to FIG. 1. The machine learning model may correspond to the machine learning model 180 or the neural network 280 of FIG. 1 or 2, and the recommendation may be determined as described above with reference to the recommendation 122 of FIG. 1 or 2.

The method 300 additionally includes providing, at 308, the recommendation by the monitoring system to the care provider.

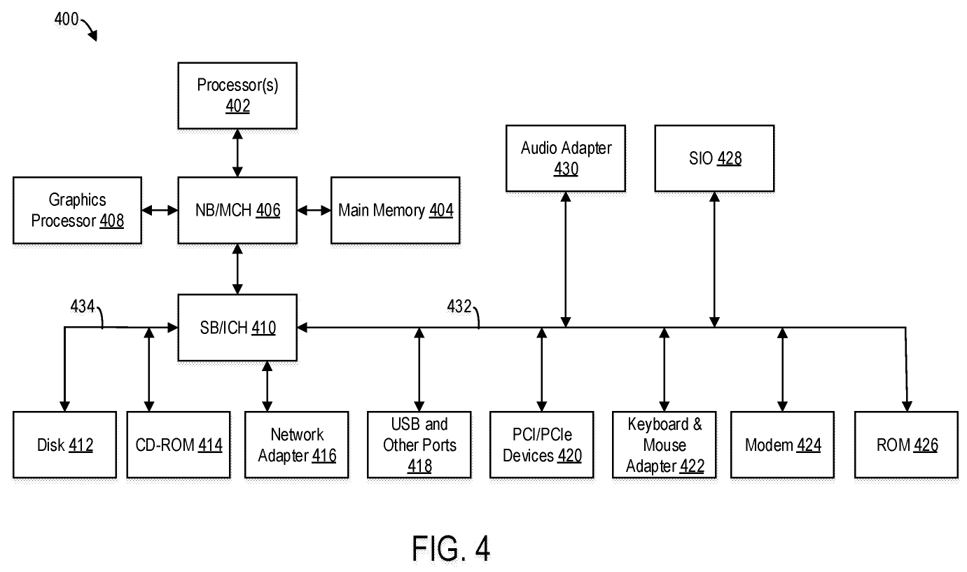

FIG. 4 is a block diagram of an example data processing system in which aspects of the illustrative embodiments may be implemented. Data processing system 400 is an example of a computer that can be applied to implement the recommendation engine 104 of FIG. 1 or the recommendation engine 204 of FIG. 2, and in which computer usable code or instructions implementing the processes for illustrative embodiments of the present disclosure may be located. In one illustrative embodiment, FIG. 4 represents a computing device that implements the recommendation engine 104 of FIG. 1 or the recommendation engine 204 of FIG. 2 augmented to include the additional mechanisms of the illustrative embodiments described hereafter.

In the depicted example, data processing system 400 employs a hub architecture including north bridge and memory controller hub (NB/MCH) 406 and south bridge and input/output (I/O) controller hub (SB/ICH) 410. Processor(s) 402, main memory 404, and graphics processor 408 are connected to NB/MCH 406. Graphics processor 408 may be connected to NB/MCH 406 through an accelerated graphics port (AGP).

In the depicted example, local area network (LAN) adapter 416 connects to SB/ICH 410. Audio adapter 430, keyboard and mouse adapter 422, modem 424, read-only memory (ROM) 426, hard disk drive (HDD) 412, compact disc read-only memory (CD-ROM) drive 414, universal serial bus (USB) ports and other communication ports 418, and peripheral component interconnect/peripheral component interconnect express (PCI/PCle) devices 420 connect to SB/ICH 410 through bus 432 and bus 434. PCI/PCle devices may include, for example, Ethernet adapters, add-in cards, and PC cards for notebook computers. PCI uses a card bus controller, while PCle does not. ROM 426 may be, for example, a flash basic input/output system (BIOS).

HDD 412 and CD-ROM drive 414 connect to SB/ICH 410 through bus 434. HDD 412 and CD-ROM drive 414 may use, for example, an integrated drive electronics (IDE) or serial advanced technology attachment (SATA) interface. Super I/O (SIO) device 428 may be connected to SB/ICH 410.

An operating system runs on processor(s) 402. The operating system coordinates and provides control of various components within the data processing system 400 in FIG. 4. In some embodiments, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 400.

In some embodiments, data processing system 400 may be, for example, an IBM® eServer™ System p® computer system, running the Advanced Interactive Executive (AIX®) operating system or the LINUX® operating system. Data processing system 400 may be a symmetric multiprocessor (SMP) system including a plurality of processors 402. Alternatively, a single processor system may be employed.

Instructions for the operating system, the object-oriented programming system, and applications or programs are located on storage devices, such as HDD 412, and may be loaded into main memory 404 for execution by processor(s) 402. The processes for illustrative embodiments of the present disclosure may be performed by processor(s) 402 using computer usable program code, which may be located in a memory such as, for example, main memory 404, ROM 426, or in one or more peripheral devices 412 and 414, for example.

A bus system, such as bus 432 or bus 434 as shown in FIG. 4, may include one or more buses. The bus system may be implemented using any type of communication fabric or architecture that provides for a transfer of data between different components or devices attached to the fabric or architecture. A communication unit, such as modem 424 or network adapter 416 of FIG. 4, may include one or more devices used to transmit and receive data. A memory may be, for example, main memory 404, ROM 426, or a cache such as found in NB/MCH 406 in FIG. 4.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a ROM, an erasable programmable read-only memory (EPROM) or Flash memory, a static random access memory (SRAM), a portable CD-ROM, a digital video disc (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or eternal storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the FIGS. illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for providing recommendations to a care provider, the method comprising:
receiving, by a monitoring system, environmental information regarding an environment in which a care provider is providing care to a recipient, wherein the environmental information includes interaction data regarding interactions between the care provider and the recipient and entity data regarding entities in the environment;
applying analytic analysis to the environmental information to generate input to a machine learning model, wherein the input includes first features indicative of aspects of the interactions and second features indicative of one or more relations between the entities;
determining a recommendation for the care provider that is predicted to facilitate achieving a goal associated with the recipient by applying the machine learning model to the input; and
providing the recommendation by the monitoring system to the care provider.

2. The method of claim 1, wherein the first features include one or more features indicative of a state of mind of the care provider or the recipient during the interactions.

3. The method of claim 2, wherein the one or more features correspond to physiological aspects of the care provider or the recipient during the interactions.

4. The method of claim 1, wherein the entities include an object, and wherein the one or more relations include a relation between the recipient and the object.

5. The method of claim 4, wherein the recommendation is to move the object.

6. The method of claim 1, wherein the interaction data includes an audio recording of the interactions, a video recording of the interactions, or an audio-visual recording of the interactions.

7. The method of claim 1, wherein the recipient is a child and the recommendation includes suggesting an activity to redirect the child or identifying a corrective discipline to be applied by the care provider.

8. The method of claim 1, wherein the recommendation is determined responsive to triggering criteria that includes detection of a pattern corresponding to a particular state of the care provider.

9. A monitoring system, comprising:
a recommendation engine configured to:
receive environmental information regarding an environment in which a care provider is providing care to a recipient, wherein the environmental information includes interaction data regarding interactions between the care provider and the recipient and entity data regarding entities in the environment;
apply analytic analysis to the environmental information to generate input to a machine learning model, wherein the input includes first features indicative of aspects of the interactions and second features indicative of one or more relations between the entities; and
determine a recommendation for the care provider that is predicted to facilitate achieving a goal associated with the recipient by applying the machine learning model to the input; and
a notification device coupled to the recommendation engine and configured to provide the recommendation to the care provider.

10. The system of claim 9, wherein the first features include one or more features indicative of a state of mind of the care provider during the interactions.

11. The system of claim 10, wherein the one or more features correspond to physiological aspects of the care provider during the interactions.

12. The system of claim 9, wherein the entities include an object, and wherein the one or more relations include a relation between the recipient and the object.

13. The system of claim 12, wherein the recommendation is to move the object.

14. The system of claim 9, wherein the interaction data includes an audio recording of the interactions, a video recording of the interactions, or an audio visual recording of the interactions.

15. The system of claim 9, wherein the recipient is a child and the recommendation includes suggesting an activity to redirect the child or identifying a corrective discipline to be applied by the care provider.

16. The system of claim 15, wherein the recommendation engine is configured to apply the machine learning model to determine the recommendation responsive to triggering criteria, and wherein the triggering criteria include detection of a pattern corresponding to a particular state of the care provider.

17. A computer readable storage medium storing computer readable program instructions that, when executed by a processor cause the processor to:
receive environmental information regarding an environment in which a care provider is providing care to a recipient, wherein the environmental information includes interaction data regarding interactions between the care provider and the recipient and entity data regarding entities in the environment;
apply analytic analysis to the environmental information to generate input to a machine learning model, wherein the input includes first features indicative of aspects of the interactions and second features indicative of one or more relations between the entities; and
determine a recommendation for the care provider that is predicted to facilitate achieving a goal associated with the recipient by applying the machine learning model to the input.

18. The computer readable storage medium of claim 17, wherein the first features include one or more features indicative of a state of mind of the care provider during the interactions.

19. The computer readable storage medium of claim 17, wherein the one or more features correspond to physiological aspects of the care provider during the interactions.

20. The computer readable storage medium of claim 17, wherein the entities include an object categorized by the machine learning model as dangerous, and wherein the one or more relations include a relation between the recipient and the object.
